(19) United States
(12) Patent Application Publication
(10) Pub. No.: US 2017/0360301 A1
(43) Pub. Date: Dec. 21, 2017

(54) CONTROL METHOD OF APPARATUS THAT DETECTS VIBRATIONS

(71) Applicant: Panasonic Intellectual Property Management Co., Ltd., Osaka (JP)

(72) Inventor: MASAAKI HARADA, Osaka (JP)

(21) Appl. No.: 15/472,419

(22) Filed: Mar. 29, 2017

(30) Foreign Application Priority Data
Jun. 15, 2016 (JP) 2016-119303

(51) Int. Cl. A61B 5/00; G01H 17/00; A61B 5/11; G06F 19/00

(52) U.S. Cl. CPC A61B 5/0051; G06F 19/3406; G01H 17/00; A61B 5/7242; A61B 5/1126; A61B 5/6892; A61B 5/4815; A61B 2560/0475

(57) ABSTRACT

A control method of an apparatus according to an aspect of the present disclosure includes acquiring first vibration data that indicates a vibration of the apparatus produced by a speaker outputting a sound. Then the control method includes storing the acquired first vibration data in a memory. Further, the control method includes acquiring second vibration data that is generated by a vibration sensor detecting a vibration including a vibration based upon a body movement of a user on bedding on which the apparatus is placed. And the control method includes generating amended data by amending the acquired second vibration data using the first vibration data stored in the memory.

### BACKGROUND

### 1. Technical Field

The present disclosure relates to a control method of an information terminal apparatus, to a body movement measuring apparatus, and to a program.

### 2. Description of the Related Art

To date, body movement measuring apparatuses have been disclosed that monitor the sleep state of a sleeping person in order to comprehend the health condition of the sleeping person and so forth.

For example, in the body movement measuring apparatus disclosed in Japanese Patent No. 4329690, sensors are installed in bedding and used to capture the lowest values of sensor values in real time, fluctuations in the lowest values of the sensor values are lessened by means of an exponential function to obtain a value that is set as a body movement determination threshold value, rough movement signals and slight movement signals are thereby separated, and the sleep state of a sleeping person is determined.

In the body movement measuring apparatus disclosed in Japanese Patent No. 4329690, it is indicated that an upper limit signal level is provided, the upper limit signal level is greater than the body movement determination threshold value and is a set multiple of the body movement determination threshold value, the body movement determination threshold value is updated on the basis of vibration data that is equal to or less than the upper limit signal level, and it is thereby possible to suppress an increase in the body movement determination threshold value in the case where the sleeping person has clearly caused a rough movement.

### SUMMARY

However, further investigation is required in order to determine body movements of a user with greater precision by means of a body movement measuring apparatus.

In one general aspect, the techniques disclosed here feature a control method of an information terminal apparatus provided with a speaker, a vibration sensor, a processor, and a memory, the control method including causing the processor to acquire self-generated vibration data that indicates a vibration of the information terminal apparatus produced by the speaker outputting a sound, store the acquired self-generated vibration data in the memory, acquire mid-sleep vibration data that is generated by the vibration sensor detecting a vibration including a vibration based upon a body movement of a user on bedding on which the information terminal apparatus is placed, and generate amended data by amending the acquired mid-sleep vibration data using the self-generated vibration data stored in the memory.

It should be noted that general or specific aspects hereof may be realized by a system, a method, an integrated circuit, a computer program, or a recording medium such as a computer-readable CD-ROM, and may be realized by an arbitrary combination of a system, a method, an integrated circuit, a computer program, and a recording medium.

According to the aforementioned aspect, further improvement can be realized.

Additional benefits and advantages of the disclosed embodiments will become apparent from the specification and drawings. The benefits and/or advantages may be individually obtained by the various embodiments and features of the specification and drawings, which need not all be provided in order to obtain one or more of such benefits and/or advantages.

### BRIEF DESCRIPTION OF THE DRAWINGS

FIG. 1 is a conceptual diagram depicting a mode of use of an information terminal apparatus in embodiment 1;

FIG. 2 is an explanatory diagram depicting a first example of vibrations detected by the information terminal apparatus;

FIG. 3 is an explanatory diagram depicting a second example of vibrations detected by the information terminal apparatus;

FIG. 4 is an explanatory diagram depicting a third example of vibrations detected by the information terminal apparatus;

FIG. 5 is an explanatory diagram depicting a fourth example of vibrations detected by the information terminal apparatus;

FIG. 6 is a block diagram depicting a hardware configuration of the information terminal apparatus in embodiment 1;

FIG. 7 is a block diagram depicting functional blocks of the information terminal apparatus in embodiment 1;

FIG. 8 is a flow diagram depicting a control method of the information terminal apparatus in embodiment 1;

FIG. 9 is a flow diagram depicting processing of a pre-measurement step in embodiment 1;

FIG. 10 is an explanatory diagram depicting the way in which the information terminal apparatus in embodiment 1 plays audio guidance for the information terminal apparatus to be placed in a stationary state;

FIG. 11 is an explanatory diagram depicting the magnitudes of vibrations detected when the information terminal apparatus in embodiment 1 plays audio guidance for the information terminal apparatus to be placed in a stationary state;

FIG. 12 is an explanatory diagram depicting the way in which the information terminal apparatus in embodiment 1 plays audio guidance for a user to turn over in bed;

FIG. 13 is an explanatory diagram depicting the magnitudes of vibrations detected when the information terminal apparatus in embodiment 1 plays audio guidance for the user to turn over in bed;

FIG. 14 is a flow diagram depicting processing of a main measurement step in embodiment 1;

FIG. 15 is a flow diagram depicting a pre-measurement step performed by the information terminal apparatus in a modified example of embodiment 1;

FIG. 16 is a block diagram depicting a functional configuration of an information terminal apparatus in embodiment 2;

FIG. 17 is a block diagram depicting a hardware configuration of an information terminal apparatus in embodiment 3;

FIG. 18 is a block diagram depicting a functional configuration of the information terminal apparatus in embodiment 3; and

FIG. 19 is a flow diagram depicting processing of a pre-measurement step in embodiment 3.

### DETAILED DESCRIPTION

### (Findings Forming the Basis for the Present Disclosure)

In recent years, it has been said that sleeping time has decreased due to changes in daily living habits in Japan, and that the level of satisfaction with sleep has declined. The sleep measuring apparatus and so forth disclosed in Japanese Patent No. 4329690 have been developed from the need that users wish to comprehend their own sleep states. However, to use the sleep measuring apparatus disclosed in Japanese Patent No. 4329690, it is necessary for special sensors to be separately purchased and attached to bedding, and it is difficult for the sleep measuring apparatus to be easily used from the aspects of cost and installation work.

Furthermore, when consideration is given to applying the body movement measuring method disclosed in Japanese Patent No. 4329690 to a body movement measuring apparatus that uses a vibration sensor such as an acceleration sensor or gyro sensor housed within a general information terminal apparatus (a smartphone, a mobile telephone terminal, or the like), the measurement precision of the sensor of the body movement measuring apparatus disclosed in Japanese Patent No. 4329690 is high, and it is therefore possible for fluctuations in the lowest values of the sensor that indicate slight movement signals of a subject to be monitored and thereby separated from rough movement signals. However, with a body movement measurement performed by an information terminal apparatus, there is variation in acquired sensor values due to differences in sensor sensitivity owing to the model of the information terminal apparatus, differences in bedding due to the information terminal apparatus being placed on bedding to measure body movements of the subject, and so forth. The sensor values acquired by the vibration sensor of the information terminal apparatus include variations in the sensor values caused by factors such as the aforementioned, and it is therefore not possible to capture slight movement signals of the subject on the basis of fluctuations in the lowest values of the sensor.

Consequently, the body movement measuring method disclosed in Japanese Patent No. 4329690 is not suitable for a body movement measuring apparatus that uses the aforementioned vibration sensor of the information terminal apparatus. Thus, for this kind of body movement measuring apparatus, there is no choice but to adopt a method in which the body movement determination threshold value is determined on the basis of fluctuations in slight movement signals having large amplitude values from among vibration data.

Furthermore, information terminal apparatuses are configured in a general-purpose manner so that various applications can be activated. Applications include those that output a sound by playing music, radio, or the like, or those that produce a vibration by operating a vibrator. Therefore, there are cases where an information terminal apparatus vibrates due to the operation of an application activated by the apparatus itself, and this vibration is detected by a vibration sensor of the information terminal apparatus. The aforementioned vibration generated by the information terminal apparatus becomes noise with respect to vibrations produced by body movements of the user to be detected by the vibration sensor of the information terminal apparatus, and become a hindrance for the body movements of the user to be accurately detected.

It is desirable to provide a body movement measuring apparatus that is able to solve these problems and to also easily determine body movements or movements of the user in a highly precise manner with only a general information terminal apparatus having a vibration sensor mounted therein owned by the user. To date, technical solutions for satisfying these demands have not been investigated.

In order to solve this kind of problem, a control method of an information terminal apparatus according to an aspect of the present disclosure is a control method of an information terminal apparatus provided with a speaker, a vibration sensor, a processor, and a memory, the control method including causing the processor to acquire self-generated vibration data that indicates a vibration of the information terminal apparatus produced by the speaker outputting a sound, store the acquired self-generated vibration data in the memory, acquire mid-sleep vibration data that is generated by the vibration sensor detecting a vibration including a vibration based upon a body movement of a user on bedding on which the information terminal apparatus is placed, and generate amended data by amending the acquired mid-sleep vibration data using the self-generated vibration data stored in the memory.

According to the aforementioned aspect, the information terminal apparatus, which serves as a body movement measuring apparatus, possesses self-generated vibration data indicating the extent to which the information terminal apparatus vibrates due to the outputting of a sound. A vibration detected by the vibration sensor is amended using the self-generated vibration data possessed, thereby suppressing the effect of a vibration generated due to a sound being output. In this way, the information terminal apparatus is able to suppress a decline in detection precision for body movements of the user, caused by a vibration generated by the information terminal apparatus outputting a sound.

For example, the self-generated vibration data includes the largest value or the average value for magnitudes of vibrations of the information terminal apparatus produced by the speaker outputting the sound, and the control method includes causing the processor to, when the mid-sleep vibration data is to be amended, generate the amended data by subtracting the largest value or the average value included in the self-generated vibration data, from the mid-sleep vibration data stored in the memory.

According to the aforementioned aspect, the information terminal apparatus possesses the magnitude of a vibration generated by the information terminal apparatus outputting a sound. By subtracting the magnitude of the aforementioned vibration from a vibration detected by the vibration sensor, a decline in detection precision for body movements of the user U can be suppressed by means of a method that can be easily implemented.

For example, an output volume of the speaker can be adjusted using a volume setting, the self-generated vibration data includes, respectively in association with a plurality of volume settings, a plurality of items of vibration data each indicating a vibration of the information terminal apparatus produced by the speaker outputting the sound at the respective volume setting, and the control method includes causing the processor to, when the mid-sleep vibration data is to be amended, generate the amended data using the vibration data associated with the volume setting for when the user is on the bedding on which the information terminal apparatus is placed, from among the plurality of items of vibration data included in the self-generated vibration data stored in the memory.

According to the aforementioned aspect, the information terminal apparatus possesses self-generated vibration data associated with a setting for the output volume of the information terminal apparatus. The effect of a vibration generated due to a sound being output can be suppressed on the basis of the vibration detected by the vibration sensor and the setting for the output volume of the information terminal apparatus in effect at that time.

For example, the control method includes causing the processor to, when the mid-sleep vibration data is to be amended, amend, using the self-generated vibration data, only the mid-sleep vibration data of a period in which the speaker was outputting the sound, from among the mid-sleep vibration data stored in the memory.

According to the aforementioned aspect, the information terminal apparatus suppresses the effect of a vibration generated by a sound being output, targeting only vibration data that is assumed to include a vibration caused by a sound that is output by the information terminal apparatus. This is because there are cases where it is not appropriate to amend the vibration data of all periods, since the effect of a sound is not included in vibration data when the information terminal apparatus is not outputting a sound.

For example, the sound includes audio that instructs the user to place the information terminal apparatus in a stationary state.

According to the aforementioned aspect, there is a benefit in that processing time can be reduced by the information terminal apparatus simultaneously carrying out the output of audio guidance for the information terminal apparatus to be placed in a stationary state, and the measurement of vibrations of the information terminal apparatus caused by the output of this audio.

For example, the control method includes, when the self-generated vibration data is to be acquired, causing the speaker to output the sound, causing the vibration sensor to detect a vibration of the information terminal apparatus produced by the speaker outputting the sound, and causing the processor to acquire vibration data generated by the vibration sensor detecting the vibration, as the self-generated vibration data.

According to the aforementioned aspect, the information terminal apparatus produces self-generated vibration data by means of the apparatus itself, and suppresses a decline in detection precision for body movements of the user on the basis of the self-generated vibration data that has been produced. By using the self-generated vibration data that is produced by the apparatus itself, the effect of a vibration caused by an output sound can be suppressed more accurately compared to using self-generated vibration data that has been produced by another apparatus, for example.

For example, the control method includes, when the self-generated vibration data is to be acquired, causing the sound to be output by a speaker of another information terminal apparatus that is different from the information terminal apparatus but is the same model as the information terminal apparatus, causing a vibration sensor of the other information terminal apparatus to detect a vibration of the other information terminal apparatus produced by the speaker of the other information terminal apparatus outputting the sound; and causing a processor of the other information terminal apparatus to acquire vibration data generated by the vibration sensor of the other information terminal apparatus detecting the vibration, as the self-generated vibration data.

According to the aforementioned aspect, the information terminal apparatus produces self-generated vibration data by means of another apparatus of the same model, and suppresses a decline in detection precision for body movements of the user on the basis of the self-generated vibration data produced by the other apparatus. By using the self-generated vibration data produced by the other apparatus of the same model, processing to produce self-generated vibration data by means of the apparatus itself becomes unnecessary. There are cases where information terminal apparatuses are manufactured on a scale of tens of thousands, for example, and therefore the effect of reducing the processing to generate self-generated vibration data in each of these tens of thousands of information terminal apparatuses is considerable.

Furthermore, a control method of an information terminal apparatus according to an aspect of the present disclosure is a control method of an information terminal apparatus provided with a vibrator, a vibration sensor, a processor, and a memory, the control method including causing the processor to acquire self-generated vibration data that indicates a vibration of the information terminal apparatus produced by the vibrator generating a vibration, store the acquired self-generated vibration data in the memory, acquire mid-sleep vibration data that is generated by the vibration sensor detecting a vibration including a vibration based upon a body movement of a user on bedding on which the information terminal apparatus is placed, and generate amended data by amending the acquired mid-sleep vibration data using the self-generated vibration data stored in the memory.

According to the aforementioned aspect, the information terminal apparatus is able to suppress a decline in detection precision for body movements of the user, caused by a vibration generated by the information terminal apparatus generating a vibration.

Furthermore, a body movement measuring apparatus according to an aspect of the present disclosure is a body movement measuring apparatus provided with a speaker, a vibration sensor, a processor, and a memory, in which the processor is made to acquire self-generated vibration data that indicates a vibration of the body movement measuring apparatus produced by the speaker outputting a sound, store the acquired self-generated vibration data in the memory, acquire mid-sleep vibration data that is generated by the vibration sensor detecting a vibration including a vibration based upon a body movement of a user on bedding on which the body movement measuring apparatus is placed, and generate amended data by amending the acquired mid-sleep vibration data using the self-generated vibration data stored in the memory.

The aforementioned aspect demonstrates an effect that is similar to that of the aforementioned control method of the information terminal apparatus.

Furthermore, a program according to an aspect of the present disclosure is a program for causing a computer to execute the aforementioned control method.

The aforementioned aspect demonstrates an effect that is similar to that of the aforementioned control method of the information terminal apparatus.

It should be noted that general or specific aspects hereof may be realized by a system, a method, an integrated circuit, a computer program, or a recording medium such as a computer-readable CD-ROM, and may be realized by an arbitrary combination of a system, a method, an integrated circuit, a computer program, or a recording medium.

Hereinafter, embodiments will be described in a specific manner with reference to the drawings.

It should be noted that the embodiments described hereinafter all represent general or specific examples. The numerical values, the shapes, the materials, the constituent elements, the arrangement positions and modes of connection of the constituent elements, the steps, and the order of the steps and the like given in the following embodiments are examples and are not intended to limit the present disclosure. Furthermore, from among the constituent elements in the following embodiments, constituent elements that are not mentioned in the independent claims indicating the most significant concepts are described as optional constituent elements.

### Embodiment 1

In the present embodiment, a body movement measuring apparatus that determines body movements of a user with greater precision, and a control method of an information terminal apparatus that operates as the body movement measuring apparatus will be described. More specifically, the body movement measuring apparatus of the present embodiment suppresses a decline in detection precision for body movements of the user, caused by vibrations generated by the body movement measuring apparatus outputting a sound.

It should be noted that the body movement measuring apparatus may have a hardware configuration that is equivalent to that of a general information terminal apparatus (a smartphone, a mobile telephone terminal, or the like) that is provided with a vibration sensor, or may be a dedicated apparatus that is provided with a general vibration sensor. Hereinafter, the case where the body movement measuring apparatus is realized by an information terminal apparatus will be described as an example.

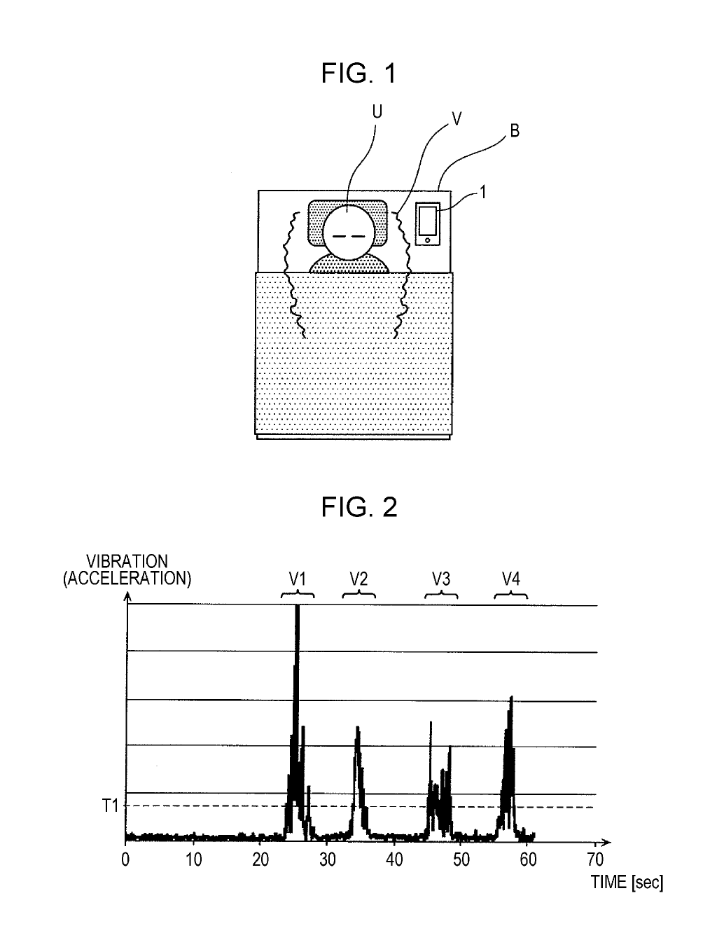

FIG. 1 is a conceptual diagram depicting a mode of use of an information terminal apparatus 1 in the present embodiment.

The information terminal apparatus 1 is placed on bedding B of a user U as depicted in FIG. 1. When the body of the user U moves, the information terminal apparatus 1 vibrates due to vibrations V of the bedding B that occur together with the movement of the body of the user U. When the information terminal apparatus 1 vibrates due to the vibrations V of the bedding B, a vibration sensor of the information terminal apparatus 1 detects those vibrations. In this way, the information terminal apparatus 1 detects whether or not there is a body movement of the user U and the magnitude of the body movement on the basis of the vibrations of the information terminal apparatus 1.

Here, the vibrations V propagate from the body of the user U, through the bedding B, and reach the information terminal apparatus 1. The way in which the vibrations V propagate differs depending on the type of bedding B (the material, weight, hardness, and the like of the contents thereof), the type of placement surface (the material, hardness, and the like) on which the bedding B is placed, and so forth. Thus, the magnitudes of the vibrations detected by the information terminal apparatus 1 are different when the bedding B is different or when the placement location or placement surface of the bedding B is different, even when the body movements by the user U have been the same. Hence, it is necessary for the information terminal apparatus 1 to measure the body movements of the user U in an appropriate manner in accordance with the actual placement location and placement surface of the bedding B.

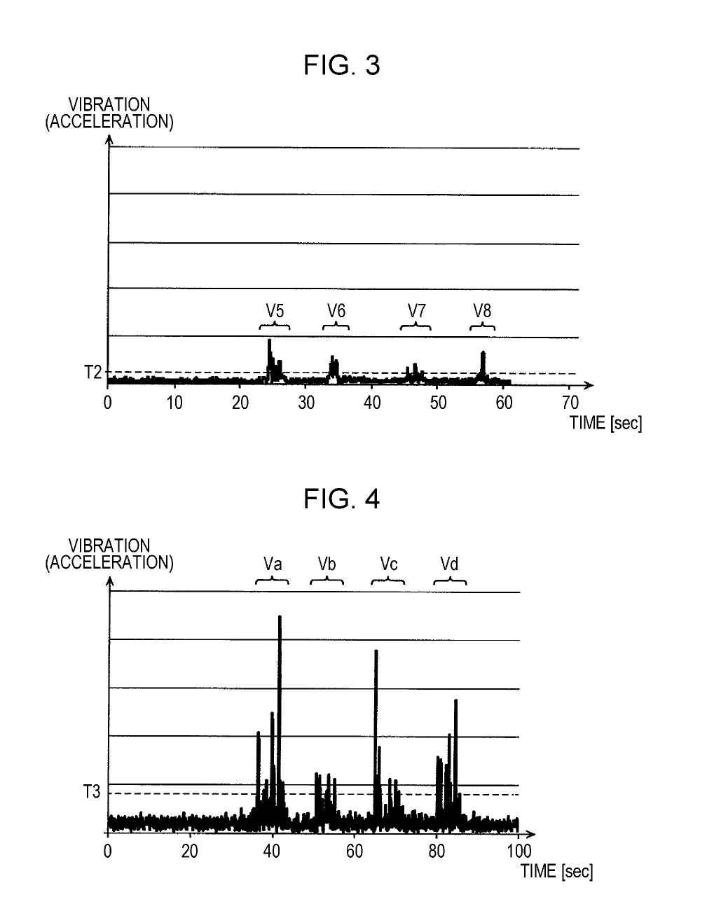

FIGS. 2 and 3 are explanatory diagrams depicting examples of vibrations detected by the information terminal apparatus 1. Specifically, FIG. 2 depicts vibrations that are produced by body movements of the user U and detected by the information terminal apparatus 1, which has been placed on a futon serving as the bedding B. FIG. 3 depicts vibrations that are produced by body movements of the user U and detected by the information terminal apparatus 1, which has been placed on a mattress serving as the bedding B. The vertical axes of the graphs in FIGS. 2 and 3 represent acceleration (absolute value of acceleration), which serves as vibration magnitude. The scale of the vertical axes of FIGS. 2 and 3 is the same.

In FIGS. 2 and 3, vibrations V1 to V8 that have a relatively large vibration magnitude (acceleration) are vibrations caused by body movements produced when the user U has turned over on the bedding B. As depicted in FIGS. 2 and 3, the magnitudes of the vibrations detected by the information terminal apparatus 1 are roughly several times different due to differences in the type of bedding B. Thus, in order to correctly detect the user U turning over in bed in either of the cases of FIG. 2 or FIG. 3, an appropriate threshold value (T1 in FIGS. 2 and T2 in FIG. 3) is provided, and turning over in bed is detected on the basis of whether a vibration (acceleration) is larger or smaller than that threshold value T3. By means of this method, it is possible to appropriately determine the user U turning over in bed in the case where the information terminal apparatus 1 does not output a sound.

However, with this method, it is not assumed at all that the information terminal apparatus 1 will output a sound. The effect of vibrations of the information terminal apparatus 1 caused by a sound being output in the case where the information terminal apparatus 1 outputs a sound will be described hereinafter.

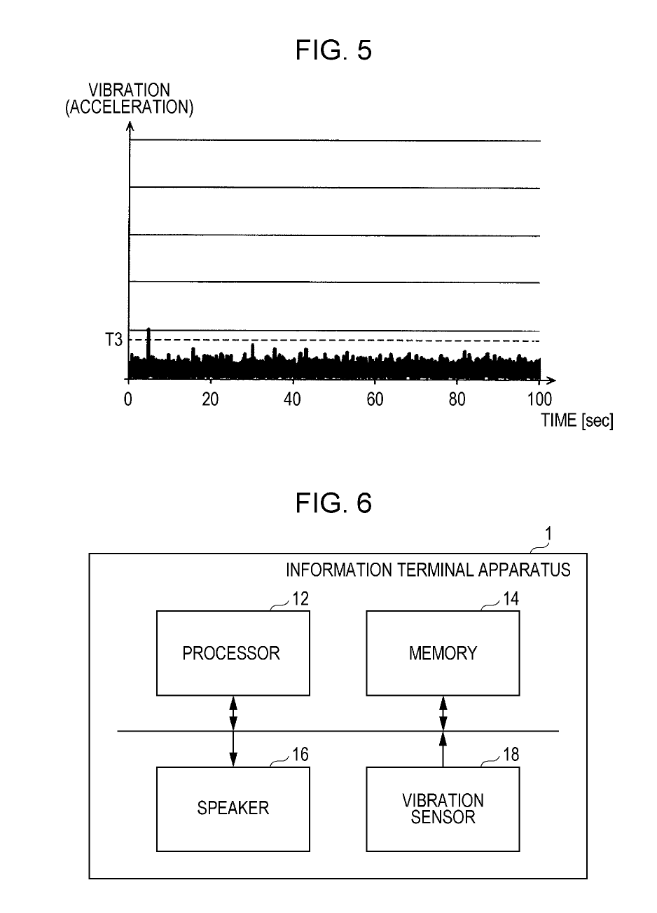

FIGS. 4 and 5 are explanatory diagrams depicting examples of vibrations detected by the information terminal apparatus 1. Specifically, FIG. 4 depicts, using acceleration, vibrations detected by the vibration sensor when the information terminal apparatus 1 is outputting a sound. Furthermore, FIG. 5 depicts, using acceleration, vibrations detected by the vibration sensor when the information terminal apparatus 1 is not outputting a sound. The scale of the vertical axes of the graphs of FIGS. 4 and 5 is the same.

It can be said that vibrations Va to Vd in FIG. 4 have waveforms similar to those of the vibrations depicted in FIGS. 2 and 3. However, actually, the vibrations Va to Vd have been obtained by the vibration sensor detecting vibrations of the information terminal apparatus 1 caused by a sound that is output by the information terminal apparatus 1. FIG. 5 depicts vibrations detected by the vibration sensor in the case where, hypothetically, the information terminal apparatus 1 does not output a sound.

In this way, when the user U turning over in bed is merely determined by determining whether the magnitude of a vibration is larger or smaller than the threshold value T3, it may be erroneously determined that the vibrations Va to Vd are the user U turning over in bed. That is, in the case where the information terminal apparatus 1 outputs a sound, the vibrations of the information terminal apparatus 1 produced by the output of the sound are erroneously determined as being vibrations that are based upon the user U turning over in bed, and therefore a problem occurs in that it is not possible to correctly determine the user U turning over in bed.

In this way, in the case where an attempt is made to detect the user U turning over in bed on the basis of vibrations detected by the information terminal apparatus 1, the detected vibrations have mixed therein vibrations caused by a sound that is output by the information terminal apparatus, and therefore there is a possibility that the user U turning over in bed may not be correctly detected. The information terminal apparatus 1 of the present disclosure implements an amendment and thereby suppresses the effect of vibrations caused by a sound that is output by the information terminal apparatus 1, from the detected vibrations, and thereby prevents a decline in detection precision. Hereinafter, a configuration and a control method of the information terminal apparatus 1 will be described in turn.

FIG. 6 is a block diagram depicting a hardware configuration of the information terminal apparatus 1 in the present embodiment.

As depicted in FIG. 6, the information terminal apparatus 1 is provided with a processor 12, a memory 14, a speaker 16, and a vibration sensor 18. The aforementioned constituent elements are connected to each other by a bus.

The processor 12 executes a control program stored in the memory 14 or the like. Functional blocks (described later on) provided in the information terminal apparatus 1 can be realized by the processor 12 executing the control program.

The memory 14 is a storage apparatus that stores information, and has a volatile storage area that is used as a work area to be used when the processor 12 executes the control program, and a nonvolatile storage area in which the control program, data, and the like are stored.

The speaker 16 is a sound-emitting apparatus that outputs sound. A vibration of the information terminal apparatus 1 can occur when the speaker 16 outputs a sound.

The vibration sensor 18 detects (or measures) vibrations of the information terminal apparatus 1, and outputs vibration data (sensor values) indicating the magnitudes of the detected vibrations. Hereinafter, a case in which the vibration sensor 18 is an acceleration sensor that detects acceleration will be described an example; however, the vibration sensor 18 may otherwise be a gyro sensor that detects angular velocity, an angle sensor that detects an angle formed with a reference angle, or the like. The vibrations detected by the vibration sensor 18 include vibrations of the information terminal apparatus 1 that are based upon vibrations propagated from the bedding B or the like when the information terminal apparatus 1 has been placed on the bedding B or the like, and vibrations of the information terminal apparatus 1 produced when the speaker 16 outputs a sound. It should be noted that the aforementioned vibrations include not only vibrations serving as periodic movements centered on one location, but also physical quantities that can be acquired by the aforementioned sensors, such as changes in location or changes in angle, for example.

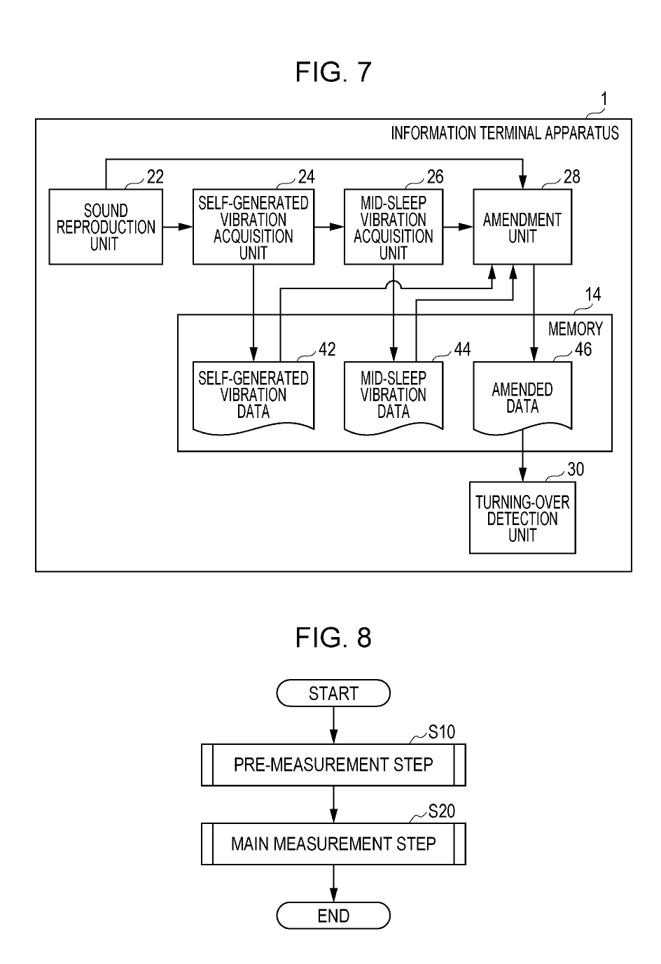

FIG. 7 is a block diagram depicting functional blocks of the information terminal apparatus 1 in the present embodiment.

As depicted in FIG. 7, the information terminal apparatus 1 is provided with a sound reproduction unit 22, a self-generated vibration acquisition unit 24, a mid-sleep vibration acquisition unit 26, an amendment unit 28, and a turning-over detection unit 30. Furthermore, the information terminal apparatus 1 possesses self-generated vibration data 42, mid-sleep vibration data 44, and amended data 46 within the memory 14.

The sound reproduction unit 22 is a processing unit that generates a source signal for a sound that is to be reproduced, and reproduces the sound using the speaker 16. The sound reproduced by the sound reproduction unit 22 is a predetermined sound such as the voice of a person, a synthesized voice, or an electronic sound. The sound reproduced by the sound reproduction unit 22 is audio guidance for prompting the user U to place the information terminal apparatus 1 in a stationary state (or to maintain a stationary state), such as “Please place the apparatus in a location that does not shake such as on a table.” or “Please wait in the current state.”, for example.

The self-generated vibration acquisition unit 24 is a processing unit that acquires the self-generated vibration data 42, which indicates vibrations of the information terminal apparatus 1 that occur due to the speaker 16 outputting a sound. The self-generated vibration acquisition unit 24 stores the acquired self-generated vibration data 42 in the memory 14. The self-generated vibration data 42 is data regarding acceleration that indicates vibration magnitude, for example. More specifically, the self-generated vibration data 42 may be time-sequential data regarding acceleration, or may include the largest value or the average value for acceleration calculated from that time-sequential data.

The self-generated vibration data 42 may be vibration data that is actually generated by the vibration sensor 18 due to the speaker 16 being made to output a predetermined sound, or may be vibration data that is generated as vibration data that would be generated by the vibration sensor 18 if the speaker 16 were made to output a predetermined sound. The latter vibration data is generated by a vibration sensor of another apparatus of the same model as the information terminal apparatus 1 due to the other apparatus causing a speaker of the other apparatus to actually output a predetermined sound, for example. Alternatively, it is also possible to use vibration data that has been appropriately revised on the basis of vibration data or the like generated for an apparatus of a similar model.

That is, the self-generated vibration acquisition unit 24 may acquire, as the self-generated vibration data 42, vibration data generated by: (1) causing a predetermined sound to be output by the speaker 16; (2) causing the vibration sensor 18 to detect a vibration of the information terminal apparatus 1 produced due to the speaker 16 outputting the predetermined sound; and (3) the vibration being detected by the vibration sensor 18.

On the other hand, the self-generated vibration acquisition unit 24 may acquire, as the self-generated vibration data 42, via a network, vibration data generated by: (1) causing a predetermined sound to be output by a speaker of another information terminal apparatus that is different from the information terminal apparatus 1 but is the same model as the information terminal apparatus 1; (2) causing a vibration sensor of the other information terminal apparatus to detect a vibration of the other information terminal apparatus produced due to the speaker of the other information terminal apparatus outputting the predetermined sound; and (3) the vibration being detected by the vibration sensor of the other information terminal apparatus.

It should be noted that it is desirable for the vibration detected by the vibration sensor 18 mentioned above to include vibrations produced due to the information terminal apparatus 1 outputting a predetermined sound, and to not include vibrations produced by other factors. Therefore, when measuring vibrations, it is assumed that the information terminal apparatus 1 is placed on a table that has been set up in such a way that vibrations produced by other factors are not propagated, or on the bedding B while the user U is not present. It should be noted that the information terminal apparatus 1 being in a stationary state, that is, being still, can be determined from vibration data detected by the vibration sensor 18. It should be noted that if the vibrations produced by other factors included in the vibrations detected by the vibration sensor 18 mentioned above are sufficiently small compared to the vibrations produced by the information terminal apparatus 1 outputting a predetermined sound, it is also possible to deem them as not being present.

The mid-sleep vibration acquisition unit 26 is a processing unit that acquires vibration data (mid-sleep vibration data 44) generated by the vibration sensor 18 detecting vibrations of the information terminal apparatus 1, including vibrations based upon body movements of the user U on the bedding B on which the information terminal apparatus 1 is placed. The mid-sleep vibration acquisition unit 26 stores the acquired mid-sleep vibration data 44 in the memory 14. The vibration data acquired by the mid-sleep vibration acquisition unit 26 includes vibration data produced while the user U is sleeping; however, it should be noted that there is no restriction thereto, and the vibration data acquired by the mid-sleep vibration acquisition unit 26 is a concept that refers to vibration data of vibrations produced when the user U is on the bedding B.

The amendment unit 28 uses the self-generated vibration data 42 to amend the mid-sleep vibration data 44 stored in the memory 14, thereby generating the amended data 46. The amendment unit 28 may generate the amended data 46 by subtracting the largest value or the average value for the magnitudes (acceleration) of the vibrations included in the self-generated vibration data 42, from the mid-sleep vibration data 44 stored in the memory 14, for example.

The turning-over detection unit 30 is a processing unit that detects the user U turning over in bed, on the basis of the amended data 46. The turning-over detection unit 30, for example, has a threshold value for acceleration, which serves as vibration, and detects vibrations having an acceleration exceeding the threshold value in the amended data 46 as vibrations produced by the user U turning over in bed. It should be noted that the turning-over detection unit 30 is not a constituent element that is essential to the present disclosure, and is an example of a processing unit that uses the amended data 46. As another example of a processing unit that uses the amended data 46, the information terminal apparatus 1 may be provided with a device control unit (not depicted) that controls an electrical device such as an air conditioner within the household of the user U by determining that the user U has left bed.

A control method of the information terminal apparatus 1 configured as mentioned above will be described hereinafter.

FIG. 8 is a flow diagram depicting a control method of the information terminal apparatus 1 in the present embodiment.

In step S10 (pre-measurement step), the information terminal apparatus 1 performs a pre-measurement to generate the amended data 46 and the like used to amend vibrations detected in a subsequent main measurement step. More specifically, the information terminal apparatus 1 performs a pre-measurement to amend the effect of vibrations of the information terminal apparatus 1 caused by the speaker 16 outputting a sound and included in the detected vibrations, and the effect of differences for each type of bedding in the vibrations detected by the vibration sensor 18.

In step S20 (main measurement step), the information terminal apparatus 1 performs a main measurement in which vibration data that includes vibrations based upon body movements of the user U while sleeping on the bedding B is acquired and the user U turning over in bed while sleeping is detected. The vibration data acquired in the main measurement step is amended using the data for amendment (self-generated vibration data) acquired in the pre-measurement step, and is then used to detect turning over in bed.

Hereinafter, the aforementioned steps will be described in detail.

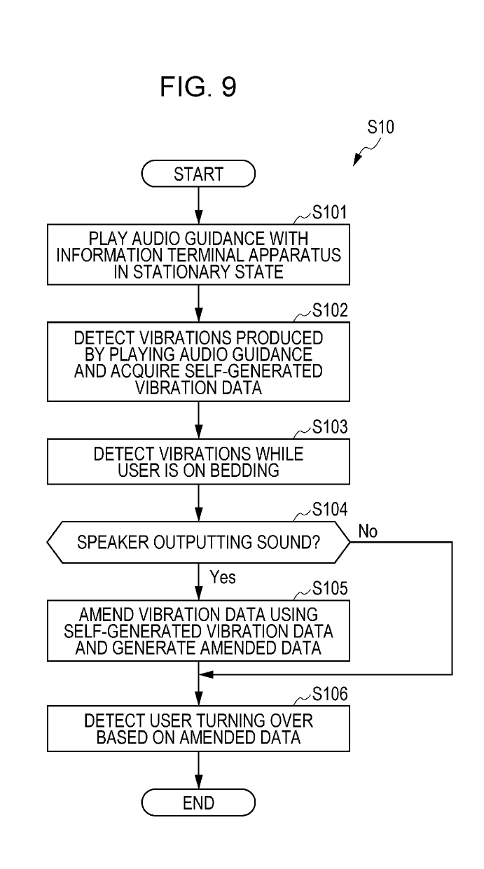

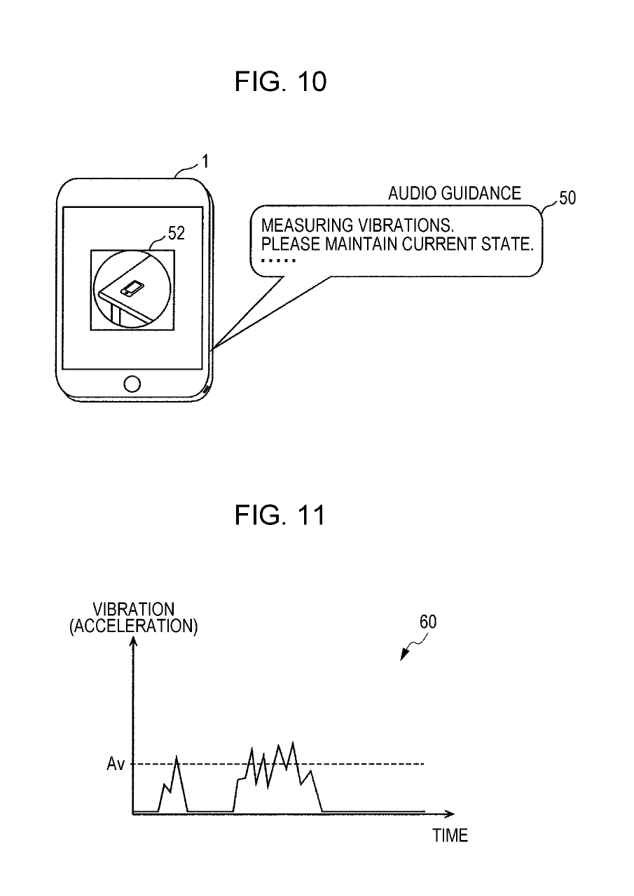

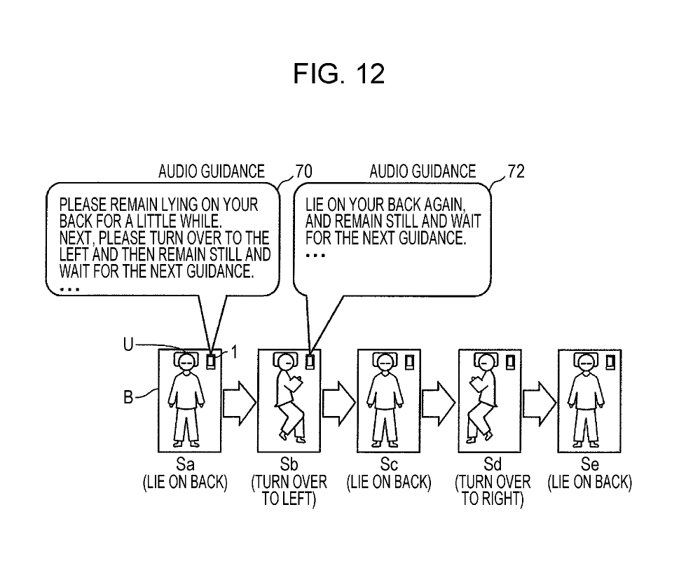

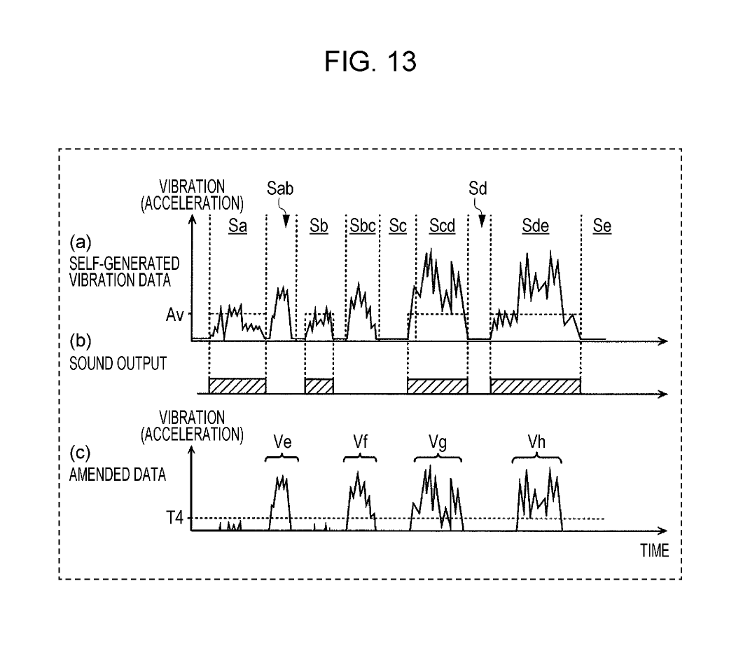

FIG. 9 is a flow diagram depicting processing of the pre-measurement step S10 in the present embodiment. FIG. 10 is an explanatory diagram depicting the way in which the information terminal apparatus 1 in the present embodiment plays audio guidance for the information terminal apparatus 1 to be placed in a stationary state. FIG. 11 is an explanatory diagram depicting the magnitudes of vibrations detected when the information terminal apparatus 1 in the present embodiment plays audio guidance for the information terminal apparatus 1 to be placed in a stationary state. FIG. 12 is an explanatory diagram depicting the way in which the information terminal apparatus 1 in the present embodiment plays audio guidance for the user U to turn over in bed. FIG. 13 is an explanatory diagram depicting the magnitudes of vibrations detected when the information terminal apparatus 1 in the present embodiment plays audio guidance for the user U to turn over in bed.

In step S101, the sound reproduction unit 22 plays audio guidance by means of the speaker 16, in a state in which the information terminal apparatus 1 has been made stationary (stationary state).

For example, the information terminal apparatus 1 plays audio guidance 50 stating “Measuring vibrations. Please maintain current state.”, as depicted in FIG. 10. By means of the audio guidance 50, the information terminal apparatus 1 prompts the user U to place the information terminal apparatus 1 in a stationary state. It should be noted that the information terminal apparatus 1 may prompt the user U in a more intuitive manner to place the information terminal apparatus 1 in a stationary state, by displaying, on a display screen, an image 52 depicting a scene in which the information terminal apparatus 1 is placed on a table. It should be noted that the aforementioned audio and image are merely examples, and other audio and images having the same purpose are permissible. The same is also true hereinafter.

In step S102, the self-generated vibration acquisition unit 24 detects, by means of the vibration sensor 18, vibrations of the information terminal apparatus 1 produced by the sound reproduction unit 22 playing the audio guidance 50 in step S101, and acquires the self-generated vibration data 42. The self-generated vibration acquisition unit 24 stores the acquired self-generated vibration data 42 in the memory 14. Furthermore, the self-generated vibration acquisition unit 24 calculates the average value for the acquired self-generated vibration data 42 and stores this in the memory 14.

For example, the self-generated vibration acquisition unit 24 acquires vibration data 60 depicted in FIG. 11 and stores this in the memory 14, and also stores an average value Av for the vibration data 60 in the memory 14.

In step S103, the mid-sleep vibration acquisition unit 26 acquires the mid-sleep vibration data 44 generated by the vibration sensor 18 detecting vibrations including vibrations based upon body movements of the user U on the bedding B on which the information terminal apparatus 1 is placed (see FIG. 13(a)).

For example, as depicted in FIG. 12, the information terminal apparatus 1 plays audio guidance 70 stating “Please remain lying on your back for a little while. Next, please turn over to the left and then remain still and wait for the next guidance.” (state Sa in FIG. 12). By means of the audio guidance 70, the information terminal apparatus 1 prompts the user U to maintain a face up position, and to then turn over to the left and maintain that position.

Furthermore, the information terminal apparatus 1 plays audio guidance 72 stating “Lie on your back again, and remain still and wait for the next guidance.” (state Sb in FIG. 12). By means of the audio guidance 72, the information terminal apparatus 1 prompts the user U to return to the face up position and maintain that position.

Thereafter, similar to the above, the information terminal apparatus 1 prompts the user U to sequentially adopt the position of having turned over to the right and the face up position (states Sc, Sd, and Se in FIG. 12). A detailed explanation is omitted here. It should be noted that FIG. 13 depicts the times at which the user U adopts the states Sa to Se depicted in FIG. 12, and also depicts states Sab, Sbc, Scd, and Sde between the aforementioned states. Here, for example, the state Sab is defined as the midway state when moving from the state Sa to the state Sb. The other states are also similarly defined.

In step S104, the amendment unit 28 determines whether or not the speaker 16 was outputting a sound when the vibration sensor 18 detected vibrations in step S103. If the speaker 16 was outputting a sound (yes in step S104), processing proceeds to step S105, and if not (no in step S104), processing proceeds to step S106. It should be noted that if the time difference between step S103 (the point in time when the vibration sensor 18 detected vibrations) and step S104 is deemed to be substantially zero (that is, steps S103 and 104 are performed at substantially the same time), the amendment unit 28 may determine whether or not the speaker 16 is outputting a sound at the point in time when the processing of step S104 is performed, instead of the aforementioned determination. Examples of a sound being output and not being output by the speaker 16 are depicted in FIG. 13(b). In FIG. 13(b), the times during which the information terminal apparatus 1 is outputting a sound are depicted using rectangles.

In step S105, the amendment unit 28 amends the vibration data detected in step S103, using the self-generated vibration data 42 acquired in step S102 and stored in the memory 14.

For example, in the case where the vibration data of FIG. 13(a) has been acquired, and a sound has been output as depicted in FIG. 13(b), the amendment unit 28 calculates the amended data 46 depicted in FIG. 13(c) by subtracting the average value Av serving as the self-generated vibration data 42 from the vibration data of the times during which a sound has been output.

In step S106, the turning-over detection unit 30 detects the user U turning over in bed on the basis of the amended data 46 generated by means of the amendment in step S105.

For example, the turning-over detection unit 30 detects vibrations Ve, Vf, Vg, and Vh having an acceleration that is larger than a predetermined threshold value T4 depicted in FIG. 13(c), as vibrations produced by the user U turning over in bed.

By means of the aforementioned series of processing, the self-generated vibration data 42 indicating vibrations of the information terminal apparatus 1 caused by the output of a sound is acquired, and the pre-measurement is completed. It should be noted that, in the aforementioned steps S101 and S102, there is a benefit in that the processing time can be reduced by simultaneously carrying out the output of the audio guidance 50 for the information terminal apparatus 1 to be placed in a stationary state, and the measurement of vibrations of the information terminal apparatus 1 caused by the output of this audio.

Furthermore, in the aforementioned explanation, the generating of the amended data 46 in step S105 was described, as an example, as being carried out with respect to only the mid-sleep vibration data 44 of periods in which the speaker 16 was outputting a sound; however, the present disclosure is not restricted thereto. For example, amendments may be carried out in all periods regardless of whether or not the speaker 16 is outputting a sound. In such a case, the determination of step S104 is not required.

Next, the processing of the main measurement step will be described.

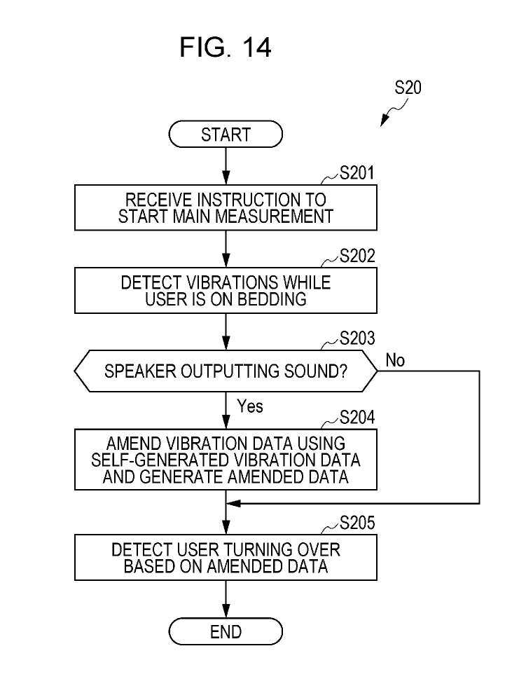

FIG. 14 is a flow diagram depicting processing of a main measurement step S20 in the present embodiment.

In step S201, the information terminal apparatus 1 receives a main measurement start instruction. For the main measurement start instruction, for example, a predetermined operation (for example, a touch operation on a touch panel screen, an operation to press a button, or the like) by the user U with respect to an input interface (not depicted) of the information terminal apparatus 1 is received as the main measurement start instruction.

In step S202, the mid-sleep vibration acquisition unit 26 acquires the mid-sleep vibration data 44 generated by the vibration sensor 18 detecting vibrations including vibrations based upon body movements of the user U on the bedding B on which the information terminal apparatus 1 is placed. The processing of step S202 is the same as the processing of step S103.

In step S203, the amendment unit 28 determines whether or not the speaker 16 was outputting a sound when the vibration sensor 18 detected vibrations in step S202. At the point in time when the processing of step S202 is performed, there is a possibility that the speaker 16 may be outputting a sound played by an audio output application such as a music playing application, a radio playing application, or the like being activated by the information terminal apparatus 1. In step S203, it is determined whether or not the speaker 16 is outputting a sound, on the basis of whether or not the information terminal apparatus 1 is activating an application capable of outputting a sound.

Thereafter, in steps S204 and S205, the generating of the amended data 46 and the detection of turning over in bed are performed by the amendment unit 28. This processing is the same as that of steps S105 and S106 in FIG. 9 and therefore a detailed explanation is omitted.

According to the aforementioned series of processing, the information terminal apparatus 1 serving as the body movement measuring apparatus of the present embodiment is able to suppress a decline in detection precision for body movements of the user U, caused by a vibration of the apparatus itself produced by the apparatus itself outputting a sound. In particular, it is possible to suppress a decline in detection precision for body movements of the user U by subtracting the effect of a vibration of the information terminal apparatus 1 caused by a sound that is output by the apparatus itself, from vibration data including the body movements of the user U.

### Modified Example of Embodiment 1

Next, a modified example will be described. In the present modified example, a body movement measuring apparatus that determines body movements of a user with greater precision, and a control method of an information terminal apparatus that operates as the body movement measuring apparatus will be described. More specifically, the body movement measuring apparatus of the present embodiment is able to further suppress a decline in detection precision for body movements of the user, by taking into consideration the volume of a sound that is output by the body movement measuring apparatus itself. It should be noted that, hereinafter, a volume setting of the information terminal apparatus, for example, is a setting for adjusting the volume that is output by the information terminal apparatus (hereinafter, the “output volume”), and can be set by means of a volume adjustment button provided in part of the casing of the information terminal apparatus, or setting processing or the like performed by the information terminal apparatus.

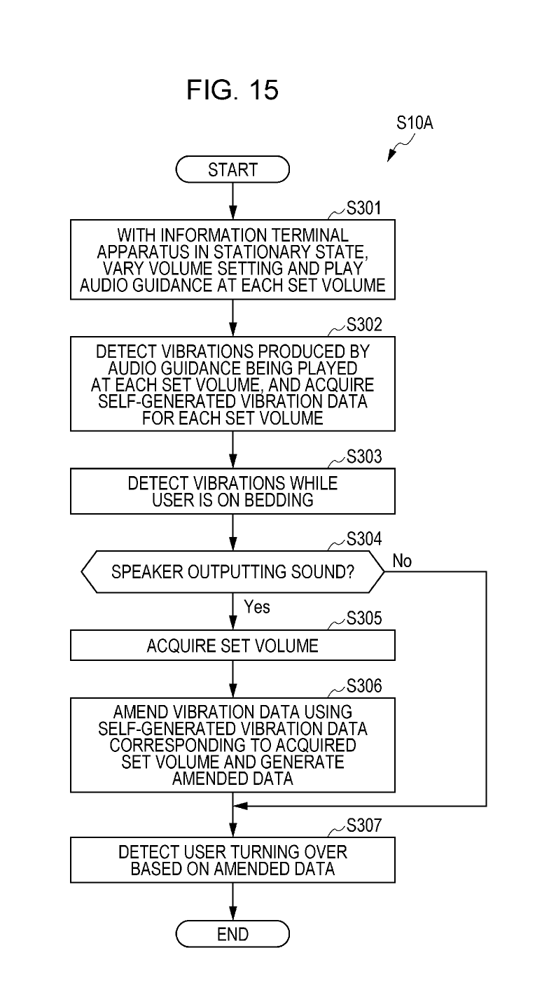

FIG. 15 is a flow diagram depicting a pre-measurement step S10A performed by the information terminal apparatus 1 in the present modified example. The pre-measurement step S10A is executed instead of the pre-measurement step S10 depicted in FIG. 9.

In step S301, the sound reproduction unit 22 plays the audio guidance 50 by means of the sound reproduction unit 22 at each set volume while changing the volume setting, in a stationary state. The volume setting mentioned here is a setting for the output volume of the information terminal apparatus 1, and is a setting that can be changed by means of an operation or the like performed by the user U.

In step S302, the self-generated vibration acquisition unit 24 detects, by means of the vibration sensor 18, vibrations of the information terminal apparatus 1 produced by the sound reproduction unit 22 playing the audio guidance 50 at each set volume in step S301, and acquires the self-generated vibration data 42. The acquired self-generated vibration data 42 includes, respectively in association with a plurality of volume settings, a plurality of items of vibration data each indicating a vibration of the information terminal apparatus 1 produced by the speaker 16 outputting the sound at the respective volume setting. The self-generated vibration acquisition unit 24 acquires the self-generated vibration data 42 for each set volume, and stores this in the memory 14.

In step S303, the mid-sleep vibration acquisition unit 26 acquires the mid-sleep vibration data 44 generated by the vibration sensor 18 detecting vibrations including vibrations based upon body movements of the user U on the bedding B on which the information terminal apparatus 1 is placed.

In step S304, the amendment unit 28 determines whether or not the speaker 16 was outputting a sound when the vibration sensor 18 detected vibrations in step S303. If the speaker 16 was outputting a sound (yes in step S304), processing proceeds to step S305, and if not (no in step S304), processing proceeds to step S307.

In step S305, the amendment unit 28 acquires the set volume for when the vibration sensor 18 detected vibrations in step S303. It should be noted that if the time difference between step S303 (the point in time when the vibration sensor 18 detected vibrations) and steps S304 and S305 is deemed to be substantially zero, the amendment unit 28 may acquire the set volume in effect at the point in time when the processing of step S305 is performed, instead of the aforementioned determination.

In step S306, the amendment unit 28 amends the vibration data detected in step S303, using the self-generated vibration data 42 associated with the set volume acquired in step S305, from among the self-generated vibration data 42 acquired in step S302 and stored in the memory 14.

In step S307, the turning-over detection unit 30 detects the user U turning over in bed on the basis of the amended data 46 generated by means of the amendment in step S306.

According to the aforementioned series of processing, the information terminal apparatus 1 serving as the body movement measuring apparatus of the present embodiment is able to suppress a decline in detection precision for body movements of the user U, by taking the set volume into consideration and subtracting the effect of a vibration of the apparatus itself caused by a sound that is output by the apparatus itself, from vibration data including the body movements of the user.

As mentioned above, the information terminal apparatus of the present embodiment possesses self-generated vibration data indicating the extent to which the information terminal apparatus vibrates due to the outputting of a sound. A vibration detected by the vibration sensor is amended using the self-generated vibration data possessed, thereby suppressing the effect of a vibration generated due to a sound being output. In this way, the information terminal apparatus is able to suppress a decline in detection precision for body movements of the user, caused by a vibration generated by the information terminal apparatus outputting a sound.

Furthermore, the information terminal apparatus possesses the magnitudes of the vibrations generated by the information terminal apparatus outputting a sound. By subtracting the magnitudes of the vibrations from vibrations detected by the vibration sensor, a decline in detection precision for body movements of the user U can be suppressed by means of a method that can be easily implemented.

Furthermore, the information terminal apparatus possesses self-generated vibration data associated with a setting for the output volume of the information terminal apparatus. The effect of a vibration generated due to a sound being output can be suppressed on the basis of the vibration detected by the vibration sensor and the setting for the output volume of the information terminal apparatus in effect at that time.

Furthermore, the information terminal apparatus suppresses the effect of a vibration generated by a sound being output, targeting only vibration data that is assumed to include a vibration caused by a sound that is output by the information terminal apparatus. This is because there are cases where it is not appropriate to amend the vibration data of all periods, since the effect of a sound is not included in vibration data when the information terminal apparatus is not outputting a sound.

Furthermore, there is a benefit in that the processing time can be reduced by the information terminal apparatus simultaneously carrying out the output of audio guidance for the information terminal apparatus to be placed in a stationary state, and the measurement of vibrations of the information terminal apparatus caused by the output of this audio.

Furthermore, the information terminal apparatus produces self-generated vibration data by means of the apparatus itself, and suppresses a decline in detection precision for body movements of the user on the basis of the self-generated vibration data that has been produced. By using the self-generated vibration data that is produced by the apparatus itself, the effect of a vibration caused by an output sound can be suppressed more accurately compared to using self-generated vibration data that has been produced by another apparatus, for example.

Furthermore, the information terminal apparatus produces self-generated vibration data by means of another apparatus of the same model, and suppresses a decline in detection precision for body movements of the user on the basis of the self-generated vibration data produced by the other apparatus. By using the self-generated vibration data produced by the other apparatus of the same model, processing to produce self-generated vibration data by means of the apparatus itself becomes unnecessary. There are cases where information terminal apparatuses are manufactured on a scale of tens of thousands, for example, and therefore the effect of reducing the processing to generate self-generated vibration data in each of these tens of thousands of information terminal apparatuses is considerable.

### Embodiment 2

In the present embodiment, a body movement measuring apparatus that determines body movements of a user with greater precision, and a control method of an information terminal apparatus that operates as the body movement measuring apparatus will be described. More specifically, the body movement measuring apparatus of the present embodiment is a body movement measuring system configured of an information terminal apparatus and another information terminal apparatus communicably connected via a network to the information terminal apparatus. It should be noted that constituent elements that are the same as those of the information terminal apparatus 1 of embodiment 1 are denoted by the same reference numerals, and detailed explanations thereof are sometimes omitted.

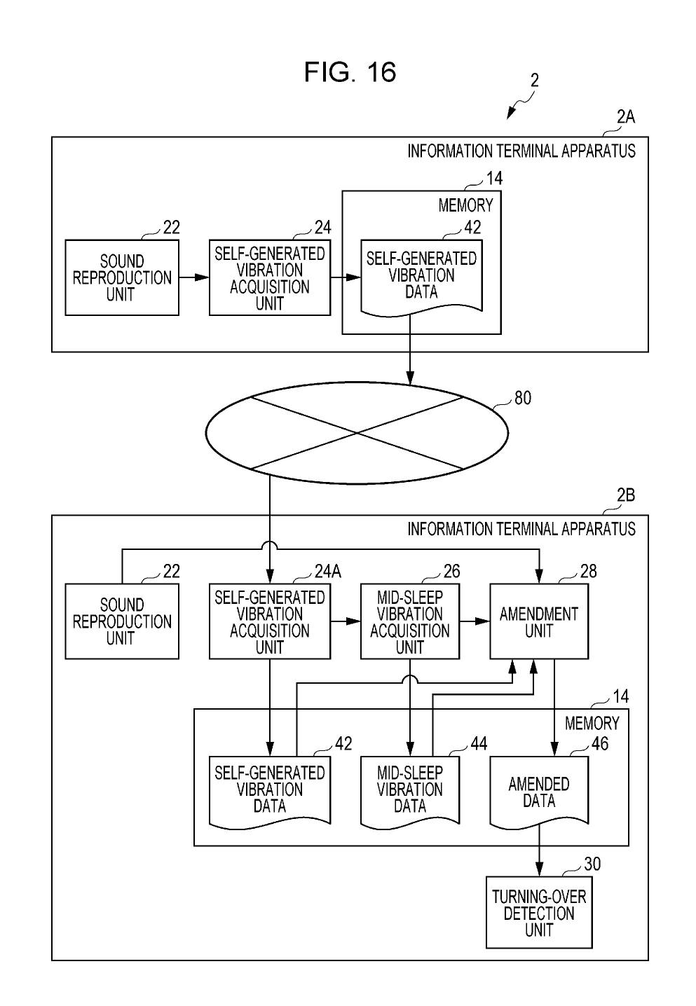

FIG. 16 is a block diagram depicting a functional configuration of a body movement measuring system 2 in the present modified example.

As depicted in FIG. 16, the body movement measuring system 2 is provided with information terminal apparatuses 2A and 2B. The information terminal apparatus 2B is placed on bedding B of a user U in the same manner as the information terminal apparatus 1 of embodiment 1. Furthermore, the information terminal apparatus 2A is another information terminal apparatus that is different from the information terminal apparatus 2B but is the same model as the information terminal apparatus 2B.

The information terminal apparatus 2A is provided with a sound reproduction unit 22 and a self-generated vibration acquisition unit 24. Furthermore, the information terminal apparatus 2A possesses self-generated vibration data 42 within a memory 14. The information terminal apparatus 2A can be said to be a configuration that is provided with only the sound reproduction unit 22, the self-generated vibration acquisition unit 24, and the memory 14 of the information terminal apparatus 1 of embodiment 1.

The sound reproduction unit 22 and the self-generated vibration acquisition unit 24 of the information terminal apparatus 2A perform the same processing as the constituent elements of the same name in the information terminal apparatus 1 of embodiment 1, thereby acquiring the self-generated vibration data 42 and storing this in the memory 14. The self-generated vibration data 42 stored in the memory 14 is provided to the information terminal apparatus 2B via a network 80.

The information terminal apparatus 2B is provided with a sound reproduction unit 22, a self-generated vibration acquisition unit 24A, a mid-sleep vibration acquisition unit 26, an amendment unit 28, and a turning-over detection unit 30. Furthermore, the information terminal apparatus 2B possesses self-generated vibration data 42, mid-sleep vibration data 44, and amended data 46 within a memory 14. The information terminal apparatus 2B is different from the information terminal apparatus 1 of embodiment 1 in being provided with the self-generated vibration acquisition unit 24A instead of the self-generated vibration acquisition unit 24.

The self-generated vibration acquisition unit 24A is a processing unit that acquires the self-generated vibration data 42 stored in the memory 14 of the information terminal apparatus 2A, via the network 80. The self-generated vibration acquisition unit 24A stores the acquired self-generated vibration data 42 in the memory 14. The amendment unit 28 and the like of the information terminal apparatus 2B generate the amended data 46 using the self-generated vibration data 42 stored in the memory 14, and turning over in bed is detected by the turning-over detection unit 30.

In this configuration, the information terminal apparatus 2B amends the acquired mid-sleep vibration data using the self-generated vibration data 42 acquired from the information terminal apparatus 2A, which is another information terminal apparatus of the same model, and therefore it is not necessary for the self-generated vibration data 42 to be generated by the information terminal apparatus 2B itself. Thus, it is possible to omit processing in which the information terminal apparatus 2B generates the self-generated vibration data 42 individually, which can contribute to reducing the processing load. The information terminal apparatuses 2A and 2B are smartphones as previously mentioned, for example, and there are cases where tens of thousands or more products of the same model are manufactured. In this case, with the information terminal apparatus 1 of embodiment 1, it is necessary for each of the tens of thousands or more information terminal apparatuses 1 to generate the self-generated vibration data 42. However, it can be said that there is no significant difference in terms of design with regard to the self-generated vibration data 42 generated in this way. In this case, with the information terminal apparatuses 2A and 2B of the present embodiment, the self-generated vibration data 42 can be generated by the information terminal apparatus 2A, which is one apparatus from among tens of thousands or more information terminal apparatuses, and the self-generated vibration data 42 that has been generated can be provided to the other information terminal apparatus 2B. Thus, there is a benefit in that the amount of processing can be reduced considerably as processing to generate the self-generated vibration data 42 in the tens of thousands of information terminal apparatuses is not required, for example.

### Embodiment 3

In the present embodiment, a body movement measuring apparatus that determines body movements of a user with greater precision, and a control method of an information terminal apparatus that operates as the body movement measuring apparatus will be described. More specifically, the body movement measuring apparatus of the present embodiment is able to suppress a decline in detection precision for body movements of the user, due to vibrations of the body movement measuring apparatus caused by vibrations output by the body movement measuring apparatus itself. It should be noted that constituent elements that are the same as those of the information terminal apparatus 1 of embodiment 1 are denoted by the same reference numerals, and detailed explanations thereof are sometimes omitted.

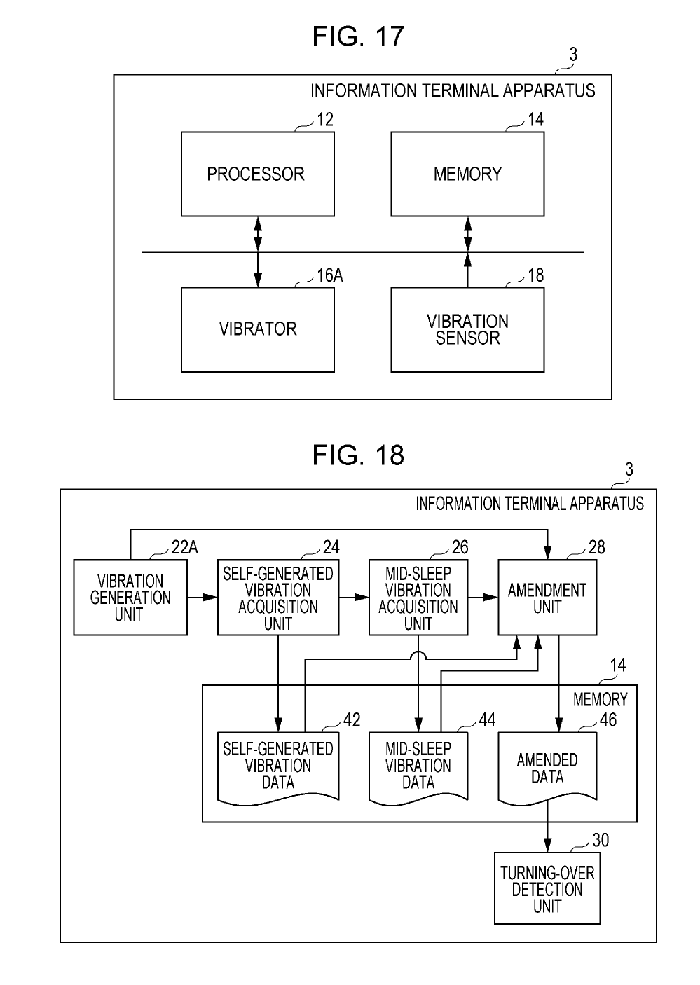

FIG. 17 is a block diagram depicting a hardware configuration of an information terminal apparatus 3 in the present embodiment.

As depicted in FIG. 17, the information terminal apparatus 3 is provided with a processor 12, a memory 14, a vibrator 16A, and a vibration sensor 18. The information terminal apparatus 3 is provided with the vibrator 16A instead of the speaker 16 of embodiment 1.

The vibrator 16A is a vibration generation apparatus (for example, a vibration motor) that generates vibrations of a predetermined intensity in accordance with control implemented by the processor 12. When the vibrator 16A generates vibrations, the information terminal apparatus 3 vibrates.

Vibrations detected by the vibration sensor 18 include vibrations of the information terminal apparatus 3 that are based upon vibrations propagated from bedding B or the like when the information terminal apparatus 3 has been placed on the bedding B or the like, and vibrations of the information terminal apparatus 3 produced when the vibrator 16A generates vibrations.

FIG. 18 is a block diagram depicting a functional configuration of the information terminal apparatus 3 in the present embodiment.

As depicted in FIG. 18, the information terminal apparatus 3 is provided with a vibration generation unit 22A, a self-generated vibration acquisition unit 24, a mid-sleep vibration acquisition unit 26, an amendment unit 28, and a turning-over detection unit 30. Furthermore, the information terminal apparatus 3 possesses self-generated vibration data 42, mid-sleep vibration data 44, and amended data 46 within the memory 14. The information terminal apparatus 3 is different from the information terminal apparatus 1 in being provided with the vibration generation unit 22A instead of the sound reproduction unit 22 of the information terminal apparatus 1. Constituent elements of the information terminal apparatus 3 that are different from the vibration generation unit 22A are the same as those of the information terminal apparatus 1 of embodiment 1 and therefore detailed explanations thereof are omitted.

The vibration generation unit 22A is a processing unit that generates a source signal indicating the strength of a vibration to be generated, and generates vibrations using the vibrator 16A. The vibrations generated by the vibration generation unit 22A are predetermined vibrations for prompting the user U to place the information terminal apparatus 3 in a stationary state (or to maintain the stationary state). The vibration generation unit 22A generates vibrations to thereby cause the information terminal apparatus 3 to vibrate, in the same manner as the sound reproduction unit 22 of embodiment 1 outputs a sound to thereby cause the information terminal apparatus 1 to vibrate.

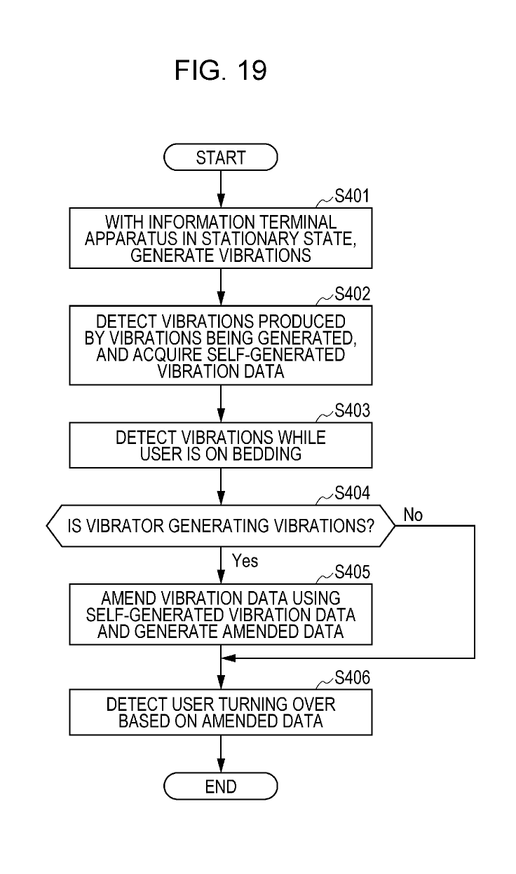

Next, the processing of the information terminal apparatus 3 will be described. FIG. 19 is a flow diagram depicting processing of a pre-measurement step in embodiment 3. It should be noted that each of the steps S401 to S406 described hereinafter can be said to be processing in which the technical content for outputting a sound and so forth in each of the steps S101 to S106 of embodiment 1 is replaced with generating a vibration and so forth. Therefore, the explanation for the steps S101 to S106 is also partially applicable to the steps S401 to S406 described hereinafter.

In step S401, the vibration generation unit 22A generates vibrations by means of the vibrator 16A, in a state in which the information terminal apparatus 3 has been made stationary (stationary state).

In step S402, the self-generated vibration acquisition unit 24 detects, by means of the vibration sensor 18, vibrations of the information terminal apparatus 3 produced by the vibration generation unit 22A generating vibrations in step S401, and acquires the self-generated vibration data 42.

In step S403, the mid-sleep vibration acquisition unit 26 acquires the mid-sleep vibration data 44 generated by the vibration sensor 18 detecting vibrations including vibrations based upon body movements of the user U on the bedding B on which the information terminal apparatus 3 is placed.

In step S404, the amendment unit 28 determines whether or not the speaker 16 was outputting a sound when the vibration sensor 18 detected vibrations in step S403. If the speaker 16 was outputting a sound (yes in step S404), processing proceeds to step S405, and if not (no in step S404), processing proceeds to step S406.

In step S405, the amendment unit 28 amends the vibration data detected in step S403, using the self-generated vibration data 42 acquired in step S402 and stored in the memory 14.

In step S406, the turning-over detection unit 30 detects the user U turning over in bed on the basis of the amended data 46 generated by means of the amendment in step S405.

According to the aforementioned series of processing, the information terminal apparatus 3 is able to suppress a decline in detection precision for body movements of the user U, as a result of vibrations of the information terminal apparatus 3 generated by the vibration generation unit 22A being detected by the vibration sensor 18.

As mentioned above, the information terminal apparatus of the present embodiment is able to suppress a decline in detection precision for body movements of the user, caused by a vibration generated by the information terminal apparatus generating a vibration.

It should be noted that, in the aforementioned embodiments, the constituent elements may be configured by using dedicated hardware, or may be realized by executing a software program suitable for the constituent elements. The constituent elements may be realized by a program execution unit such as a CPU or a processor reading out and executing a software program recorded in a recording medium such as a hard disk or a semiconductor memory. Here, software that realizes the information terminal apparatus and the like of the aforementioned embodiments is a program such as the following.

In other words, this program causes a computer to execute a control method of an information terminal apparatus provided with a speaker, a vibration sensor, a processor, and a memory, the control method including causing the processor to acquire self-generated vibration data that indicates a vibration of the information terminal apparatus produced by the speaker outputting a sound, store the acquired self-generated vibration data in the memory, acquire mid-sleep vibration data that is generated by the vibration sensor detecting a vibration including a vibration based upon a body movement of a user on bedding on which the information terminal apparatus is placed, and generate amended data by amending the acquired mid-sleep vibration data using the self-generated vibration data stored in the memory.

An information terminal apparatus and the like according to one or more aspects has been described hereinabove on the basis of the embodiments; however, the present disclosure is not restricted to these embodiments. Modes in which various modifications conceived by a person skilled in the art have been implemented in the present embodiments, and modes constructed by combining the constituent elements in different embodiments may also be included within the scope of one or more aspects provided they do not depart from the purpose of the present disclosure.

The present disclosure can be used for a body movement measuring apparatus that measures body movements, a control apparatus that controls another device on the basis of the detection of body movements, and the like.

What is claimed is:

1. A control method of an apparatus including a speaker, a vibration sensor, a processor, and a memory, the control method comprising:
acquiring, using the processor and the vibration sensor, first vibration data that indicates a vibration of the apparatus produced by the speaker outputting a sound;
storing, using the processor, the acquired first vibration data in the memory;
acquiring, using the processor and the vibration sensor, second vibration data that is generated by the vibration sensor detecting a vibration including a vibration based upon a body movement of a user on bedding on which the apparatus is placed; and
generating, using the processor, amended data by amending the second vibration data using the stored first vibration data.

2. The control method according to claim 1,
wherein the first vibration data includes a largest value or an average value for magnitudes of vibrations of the apparatus produced by the speaker outputting the sound,
and the control method further includes:
storing, using the processor, the acquired second vibration data in the memory; and,
when the second vibration data is to be amended, generating, using the processor, the amended data by subtracting the largest value or the average value included in the first vibration data, from the stored second vibration data.

3. The control method according to claim 1,
wherein an output volume of the speaker can be adjusted using a volume setting,
the first vibration data includes, respectively in association with a plurality of volume settings, a plurality of items of vibration data each indicating a vibration of the apparatus produced by the speaker outputting the sound at the respective volume setting,
and the control method further includes:
when the second vibration data is to be amended, generating, using the processor, the amended data using the vibration data associated with the volume setting for when the user is on the bedding on which the apparatus is placed, from among the plurality of items of vibration data included in the stored first vibration data.

4. The control method according to claim 1,
wherein the control method further includes:
storing, using the processor, the acquired second vibration data in the memory; and,
when the second vibration data is to be amended, amending, using the processor and using the first vibration data, only the second vibration data of a period in which the speaker was outputting the sound, from among the stored second vibration data.

5. The control method according to claim 1,
wherein the sound includes audio that instructs the user to place the apparatus in a stationary state.

6. The control method according to claim 1,
wherein the control method includes, when the first vibration data is to be acquired:
outputting the sound, using the speaker;
detecting, using the vibration sensor, a vibration of the apparatus produced by the speaker outputting the sound; and
acquiring, using the processor, vibration data generated by the vibration sensor detecting the vibration, as the first vibration data.

7. The control method according to claim 1,
wherein the control method includes, when the first vibration data is to be acquired:
outputting the sound, using a speaker of another apparatus that is different from the apparatus but is the same model as the apparatus;
detecting, using a vibration sensor of the other apparatus, a vibration of the other apparatus produced by the speaker of the other apparatus outputting the sound; and
acquiring, using a processor of the other apparatus, vibration data generated by the vibration sensor of the other apparatus detecting the vibration, as the first vibration data.

8. A control method of an apparatus including a vibrator, a vibration sensor, a processor, and a memory, the control method comprising:
acquiring, using the processor and the vibration sensor, first vibration data that indicates a vibration of the apparatus produced by the vibrator generating a vibration;
storing, using the processor, the first vibration data in the memory;
acquiring, using the processor and the vibration sensor, second vibration data that is generated by the vibration sensor detecting a vibration including a vibration based upon a body movement of a user on bedding on which the apparatus is placed; and
generating, using the processor, amended data by amending the second vibration data using the first vibration data.

9. An apparatus comprising:
a speaker;
a vibration sensor;
a processor; and
a memory storing thereon a computer program, which when executed by the processor, causes the processor to perform operations including:
acquiring, using the vibration sensor, first vibration data that indicates a vibration of the apparatus produced by the speaker outputting a sound;
storing the first vibration data in the memory;
acquiring, using the vibration sensor, second vibration data that is generated by the vibration sensor detecting a vibration including a vibration based upon a body movement of a user on bedding on which the apparatus is placed; and
generating amended data by amending the second vibration data using the first vibration data.

10. A non-transitory recording medium storing thereon a computer program, which when executed by a processor, causes the processor to perform operations according to claim 1.
